(19) United States
(12) Patent Application Publication
(10) Pub. No.: US 2024/0194356 A1
(43) Pub. Date: Jun. 13, 2024

(54) MEDICAL DOCUMENT ANALYSIS METHOD AND SYSTEM THEREOF

(71) Applicant: ASG Inspiration Laboratory Ltd., Mid-Level West (HK)

(72) Inventors: Johnson LEE, Mid-Level West (HK); Jao Juen HUNG, Mid-Level West (HK); Sung Tsai YU, Mid-Level West (HK); Shih Pan CHAO, Mid-Level West (HK); Hao-Wei HUANG, Mid-Level West (HK)

(73) Assignee: ASG Inspiration Laboratory Ltd., Mid-Level West (HK)

(21) Appl. No.: 18/319,842

(22) Filed: May 18, 2023

(30) Foreign Application Priority Data
Dec. 9, 2022 (TW) 111147454

(51) Int. Cl. G16H 70/00; G06F 16/33; G16H 50/70

(52) U.S. Cl. CPC G16H 70/00; G06F 16/3334; G16H 50/70

(57) ABSTRACT

A medical document analysis method includes following steps: performing recursive search based on keywords extracted from first medical documents to obtain second medical documents; analyzing feature labels of the second medical documents; projecting the second medical documents onto a multi-dimensional map according to the feature labels; estimating second symptoms from a first symptom; selecting third medical documents based on the first symptom and the second symptom from the multi-dimensional map; analyzing correlation between the third medical documents and their respective feature labels in the multi-dimensional map to form a label topology map; and selecting a target branch path from branch paths in the label topology map, and displaying information about the target branch path.

### CROSS-REFERENCE TO RELATED APPLICATION

This application claims priority to Taiwan Application Serial Number 111147454, filed Dec. 9, 2022, which is herein incorporated by reference in its entirety.

### BACKGROUND

### Field of Invention

The present disclosure relates to a medical literature analysis method and related system. More particularly, the present disclosure relates to an analysis method and system for searching for valuable target medical literature based on natural language processing and neural networks.

### Description of Related Art

Every year, a large number of medical researches and literatures are published in various journals, papers, and medical forums. These publications contain data from clinical studies or laboratory simulations on drug use, medical effects, side effects, and user populations. This data usually describes specific research purposes, such as targeting specific diseases, patient populations, medication methods, medication frequency, expected effects, actual effects, and control group data.

Further research and analysis of these medical literatures can often lead to medically valuable discoveries within or even outside the original scope of these medical literatures. However, in practice, the number of these medical literatures is huge and the experimental subjects and conclusions involved are different. Without an efficient way to perform automated analysis, it is difficult for researchers to quickly find valuable references among them.

### SUMMARY

The disclosure provides a medical document analysis method. The medical document analysis method comprises following steps: performing a recursive search based on keywords of a plurality of first medical documents to obtain a plurality of second medical documents; analyzing contents of the plurality of second medical documents, and marking each of the plurality of second medical documents with a plurality of feature labels; projecting the plurality of second medical documents onto different coordinate positions in a multi-dimensional map respectively based on the at least one feature label of each of the plurality of second medical documents, wherein dimensions of the multi-dimensional map correspond to the plurality of feature labels, and distances between the coordinate positions correspond to a correlation between the plurality of second medical documents in response to selecting a first symptom, estimating a plurality of second symptoms related to the first symptom; selecting a plurality of coordinates matching the first symptom and the plurality of second symptoms from the multi-dimensional map, and selecting a plurality of third medical documents from the plurality of second medical documents based on a node adjacent to the plurality of coordinates; analyzing a correlation between the plurality of third medical documents and the at least one feature label of each of the plurality of third medical documents in the multi-dimensional map to form a label topology map, wherein a top layer node in the label topology map is the first symptom, the label topology map comprises a plurality of branch paths, and each of the plurality of branch paths starts from the first symptom and passes through a drug formulation, a receptor name and a drug dosage form; and selecting a target branch path from the plurality of branch paths, and displaying information related to the target branch path.

The disclosure further provides a medical document analysis system. The medical document analysis system comprises a communication transceiver unit, a user interface, and a process unit. The communication transceiver is communicatively connected to an academic database. The process unit is coupled to the communication transceiver unit and the user interface. The process unit is configured to perform a recursive search based on keywords of a plurality of first medical documents to obtain a plurality of second medical documents. The process unit is further configured to analyze contents of the plurality of second medical documents, and mark each of the plurality of second medical documents with a plurality of feature labels. The process unit is further configured to project the plurality of second medical documents onto different coordinate positions in a multi-dimensional map respectively based on the at least one feature label of each of the plurality of second medical documents, wherein dimensions of the multi-dimensional map correspond to the plurality of feature labels, and distances between the coordinate positions correspond to a correlation between the plurality of second medical documents. In response to selecting a first symptom, the process unit is further configured to estimate a plurality of second symptoms related to the first symptom. The process unit is further configured to select a plurality of coordinates matching the first symptom and the plurality of second symptoms from the multi-dimensional map, and select a plurality of third medical documents from the plurality of second medical documents based on a node adjacent to the plurality of coordinates. The process unit is further configured to analyze a correlation between the plurality of third medical documents and the at least one feature label of each of the plurality of third medical documents in the multi-dimensional map to form a label topology map, wherein a top layer node in the label topology map is the first symptom, the label topology map comprises a plurality of branch paths, and each of the plurality of branch paths starts from the first symptom and passes through a drug formulation, a receptor name, and a drug dosage form. The process unit is further configured to select a target branch path from the plurality of branch paths, and display information related to the target branch path.

It is to be understood that both the foregoing general description and the following detailed description are by examples, and are intended to provide further explanation of the disclosure as claimed.

### BRIEF DESCRIPTION OF THE DRAWINGS

The disclosure can be more fully understood by reading the following detailed description of the embodiment, with reference made to the accompanying drawings as follows:

FIG. 1 is a function block diagram illustrating a medical document analysis system according to some embodiments of the present disclosure.

FIG. 2 is a flow diagram illustrating a medical document analysis method according to some embodiments of the present disclosure.

FIG. 3 is a flow diagram illustrating detailed steps of one of the steps shown in FIG. 2 according to some embodiments of the present disclosure.

FIG. 4 is a flow diagram illustrating detailed steps of another step shown in FIG. 2 according to some embodiments of the present disclosure.

FIG. 5 is a schematic diagram illustrating second medical documents in a multi-dimensional map according to some embodiments of the present disclosure.

FIG. 6 is a flow diagram illustrating detailed steps of one of the steps shown in FIG. 2 according to some embodiments of the present disclosure.

FIG. 7 is a schematic diagram illustrating a label topology map according to some embodiments of the present disclosure.

### DETAILED DESCRIPTION

Reference will now be made in detail to the present embodiments of the disclosure, examples of which are illustrated in the accompanying drawings. Wherever possible, the same reference numbers are used in the drawings and the description to refer to the same or like parts.

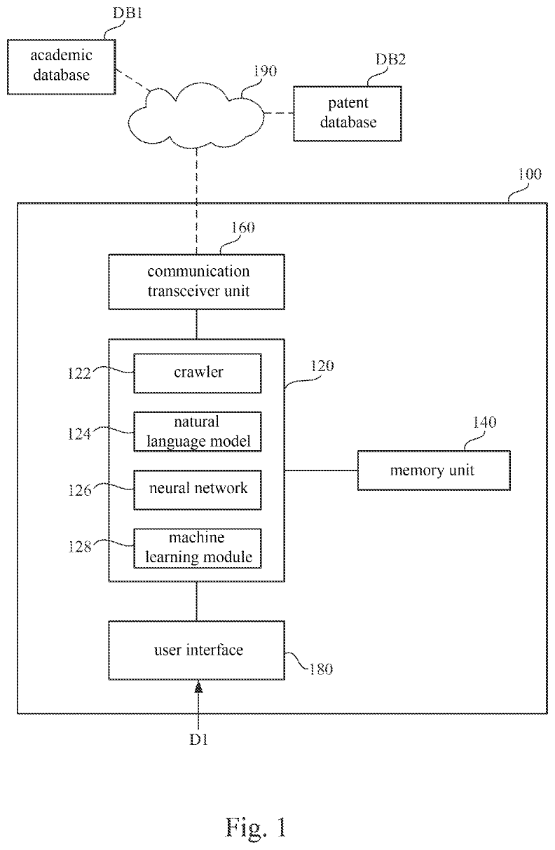

Reference is made to FIG. 1. FIG. 1 is a function block diagram illustrating a medical document analysis system 100 according to some embodiments of the present disclosure. In an embodiment, the medical document analysis system 100 is configured to perform a feasibility study analysis for drug. For example, the medical document analysis system 100 can search for the previous medical documents or the published medical journals related to approved drugs, analyze whether the approved drugs or drug ingredients in the experiment have a different use that is not currently discovered or approved and estimate the feasibility and potential commercial value of the new use. On the other hand, in the phase of developing new drugs, the medical document analysis system 100 can search for the medical documents mentioned drugs with similar use. If experimental results with discrepancy are found in the previous medical documents, significant difference exists between the previous medical documents and other literatures, or the previous medical documents are conflict or inconsistent with current experimental results, the developers can find out the reason by further experiments based on the medical documents in the shortest time.

As shown in FIG. 1, the medical document analysis system 100 comprises a process unit 120, a memory unit 140, a communication transceiver unit 160, and a user interface 180.

In an embodiment, the medical document analysis system 100 can be a server, a computer, a smart phone, a network switch, a gateway, a router, or device with communication functions. In the embodiment shown in FIG. 1, the medical document analysis system 100 can also be composed of distributed computing systems, that is, the process unit 120, the memory unit 140, the communication transceiver unit 160, and the user interface 180 of the medical document analysis system 100 can be distributed across multiple electronic devices connected via an internet 190, and the multiple electronic devices process analytical computing tasks separately. The medical document analysis system 100 can receive or transmit network packets to the internet 190 by the communication transceiver unit 160. Accordingly, the medical document analysis system 100 can obtain data in an academic database DB1 and a patent database DB2 on the internet 190.

In an embodiment, the communication transceiver unit 160 can comprise an Ethernet transceiver circuit, a Wi-Fi transceiver circuit, a Bluetooth transceiver circuit, or other communication transceiver circuit. In some embodiments, the communication transceiver unit 160 can be implemented by hardware of above-mentioned transceiver circuits or communication interfaces. In other embodiments, the communication transceiver unit 160 can also be implemented by logic circuit of system on a chip (SoC) executing software/firmware related to above-mentioned communication functions.

The process unit 120 of the medical document analysis system 100 is configured to process the data computing task on the medical document analysis system 100, such as the process unit 120 can execute a crawler 122, a natural language model 124, a neural network 126, and a machine learning module 128. In an embodiment, the process unit 120 can be implemented by a processor, a central processing unit (CPU), a graphic processing unit (GPU), a tensor processing unit (TPU), an application specific integrated circuit (ASIC), or other similar process unit. The crawler 122 executed by the process unit 120 connects to the academic database DB1 and the patent database DB2 on the internet 190 by the communication transceiver unit 160. The crawler 122 is configured to search and download various medical documents from the academic database DB1, such as medical research papers, news, journals and new drug application documents form drug administration, etc. On the other hand, the crawler 122 is configured to search and download various patent documents from the patent database DB2, such as patent applications, specifications of patent publications, specifications of granted patent publications, patent office action responses history journal, patent assignment documents, and patent fee notices, etc.

In an embodiment, the natural language model 124 is configured to process natural language (e.g., semantic recognition, keyword extraction, and article abstract extraction, etc.), and the neural network 126 is configured to simulate and valuate the reference value and the success rate of medical documents. The machine learning module 128 is configured to train the natural language model 124 and the neural network 126 in the training phase to determine the weights and the parameters of the natural language model 124 and the neural network 126 during execution.

The memory unit 140 is configured to store parameters, software, codes, or instruction sets of the crawler 122, the natural language model 124, the neural network 126, and the machine learning module 128 mentioned above. In an embodiment, the process unit 120 reads the above-mentioned parameters, software, codes, or instruction sets and executes them. The memory unit 140 comprises computer-readable storage medium such as hard disk, flash memory, static memory, dynamic memory, or cache memory, etc.

In an embodiment, the user interface 180 can comprise a monitor, a keyboard, a mouse, a trackpad, a microphone, or a speaker, etc. The user interface 180 is configured to display, play, or show information content and provide the user of the medical document analysis system 100 to input instructions, operations, or information via the user interface 180.

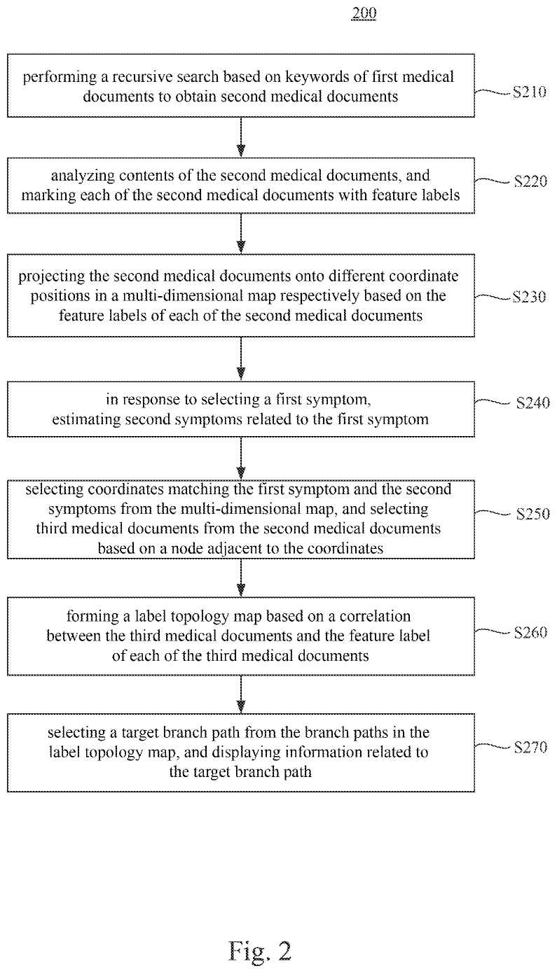

Reference is also made to FIG. 2. FIG. 2 is a flow diagram illustrating a medical document analysis method 200 according to some embodiments of the present disclosure, the medical document analysis method 200 can be executed by the medical document analysis system 100 shown in FIG. 1.

First, when a user (e.g., developers in pharmaceutical companies) wants to start a medical development for specific symptom, for example, new drug development or research for whether existing drugs can solve the symptom, the user can search for medical documents related to current research, for example, the user can collect medical documents related to diabetes from recent journals in order to develop drug for solving diabetes, and the medical documents collected can be taken as first medical documents D1. The user can take the first medical documents D1 as an input signal and input the input signal into the medical document analysis system 100 via the user interface 180 (or the communication transceiver unit 160). In the meantime, the medical document analysis system 100 can execute the medical document analysis method 200, analyze the first medical documents D1 inputted by the user, provide suggested direction of development, and search for related valuable medical documents automatically. The detail analysis and suggested method of the medical document analysis system 100 and the medical document analysis method 200 are further illustrated in the following paragraphs.

As shown in FIG. 1 and FIG. 2, first, the process unit 120 executes step S210, performing a recursive search based on a plurality of keywords of a plurality of first medical documents to obtain a plurality of second medical documents.

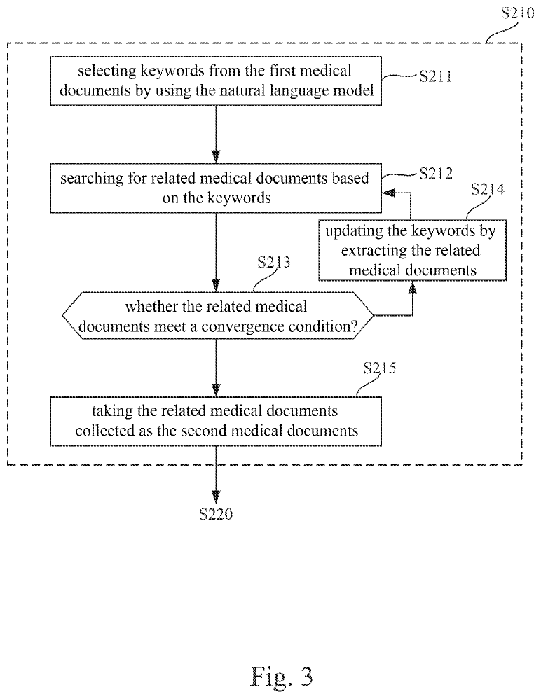

Reference is also made to FIG. 3. FIG. 3 is a flow diagram illustrating detailed steps of step S210 shown in FIG. 2 according to some embodiments of the present disclosure. As shown in FIG. 3, step S210 further comprises step S211-S215. First, the process unit 120 executes step S211, selecting a plurality of keywords from the headings, subheadings, abstracts, or the whole contents of the first medical documents D1 by using the natural language model 124. In an embodiment, the natural language model 124 can analyze the meaning of the phrases, the relevance of the context, the importance of the phrases, and the frequency of the phrases in the first medical documents D1. For example, the natural language model 124 can set a phrase with high frequency (e.g., a phrase appears more than 10 times) as a keyword; the natural language model 124 can set a phrase appeared in the headings or the abstracts as a keyword; and the natural language model 124 can set a phrase of symptoms, curative effects, diseases, drug names, drug principles, or experimental methods as a keyword.

In an embodiment, the natural language model 124 can establish and store related phrase dictionaries for symptoms or diseases to be studied to avoid interpretation error during the natural language process of the first medical documents D1.

Next, the process unit 120 executes step S212, searching the academic database DB1 by using the crawler 122 based on the plurality of keywords to obtain a plurality of related medical documents. The crawler 122 can be implemented by robotic process automation (RPA) code. The crawler 122 will simulate normal internet users to access the academic database DB1, search and obtain the related medical documents including medical research papers, news, journals, and new drug applications from drug administration from various countries.

In some embodiments, the crawler 122 can fully automate the search and download of the related medical documents. In other embodiments, since the academic database DB1 storing journals and papers may need to pay before access or have a robot recognition function, it needs to be cracked manually (e.g., the user input account, password, or authentication code via the user interface 180) or supplemented by optical recognition.

After that, the process unit 120 executes step S213, determining whether the plurality of related medical documents collected meet a preset convergence condition. For example, the convergence condition can be set as whether the number of the related medical documents collected has reached 500.

If the convergence condition is not met (e.g., the number of the related medical documents collected is only 30), the process unit 120 executes step S214, updating the plurality of keywords by extracting the 30 related medical documents by using the natural language model 124. At this point, the updated keyword set comprises keywords extracted from the first medical documents D1 and keywords extracted from the 30 related medical documents. Subsequently, the process unit 120 executes step S212 again, searching for the related medical documents based on the expanded keywords until the convergence condition is met.

The convergence condition mentioned above is related to the number of the related medical documents, but the present disclosure is not limited thereto. In other embodiments, the convergence condition can be set as the computing time for searching the related medical documents (e.g., 5 minutes), the number of repeated searches (e.g., expanded search for 3 times), or other equivalent convergence condition. In other embodiments, the related medical documents collected and the corresponding keywords are displayed on the user interface 180, and the user can determine whether the convergence condition is met accordingly.

On the other hand, if the convergence condition is met, the process unit 120 executes step S215, taking all of the plurality of related medical documents collected as the plurality of second medical documents for subsequent analysis.

It is noted that, in some embodiments, after the step S211 mentioned above, and before the step S212, the medical document analysis system 100 and the medical document analysis method 200 can allow the user add a new keyword to the above-mentioned keyword sets and remove a excluded keyword from the above-mentioned keyword sets manually via the user interface 180.

Also, the medical document analysis system 100 and the medical document analysis method 200 can assign a plurality of weights to each of the plurality of keywords. For example, the keywords can be distinguished as necessary keywords, unnecessary keywords, and non-existent keywords (i.e., excluded keywords), wherein the highest weight can be assigned to the necessary keywords, and weights ranging from high to low can be assigned to each of the unnecessary keywords. The weight assignment mentioned above can be executed by the natural language model 124 based on the importance of the keywords automatically, or executed by the user via the user interface 180 manually. In this case, the plurality of related medical documents are searched based on the weighted keywords in step S212.

As shown in FIG. 1 and FIG. 2, the process unit 120 executes step S220, analyzing contents of the plurality of second medical documents, and marking each of the plurality of second medical documents with a plurality of feature labels. In an embodiment, the feature labels labeled comprise a published time, a publication, a drug name label, a symptom label, an author label, a drug receptor label, a dosage form label, a drug composition or an active pharmaceutical ingredient (API), a side effect, a pharmacological mechanism (e.g., blocking method, path, receptor, replication stopping), etc.

As shown in FIG. 1 and FIG. 2, the process unit 120 executes step S230, projecting the plurality of second medical documents onto different coordinate positions in a multi-dimensional map respectively based on the plurality of feature labels of each of the plurality of second medical documents.

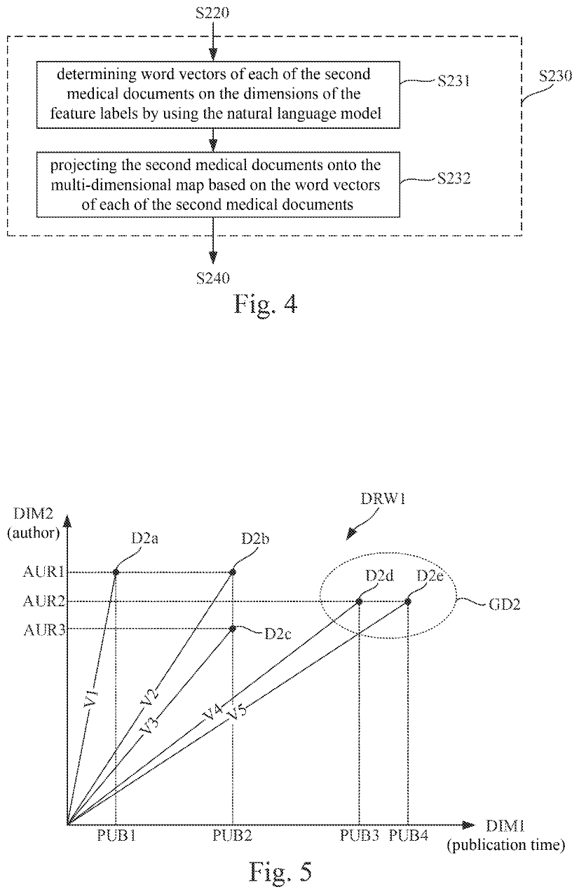

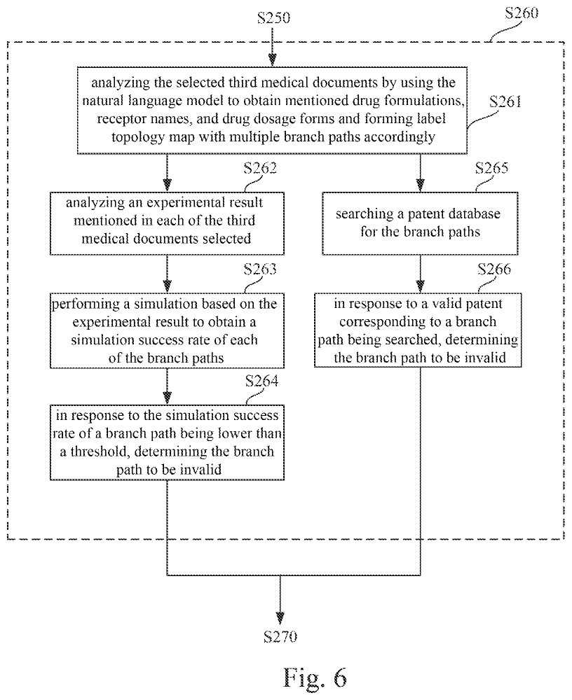

References are also made to FIG. 4 and FIG. 5. FIG. 4 is a flow diagram illustrating detailed steps of step S230 shown in FIG. 2 according to some embodiments of the present disclosure. FIG. 5 is a schematic diagram illustrating five second medical documents D2a-D2e in a multi-dimensional map DRW1 according to some embodiments of the present disclosure.

For the convenience of explanation, the dimensions of the multi-dimensional map DRW1 have been simplified, and the two dimensions DIM1 and DIM2 shown in FIG. 5 are two of the dimensions of the multi-dimensional map DRW1. In this case, the dimension DIM1 of the multi-dimensional map DRW1 is author, and another dimension DIM2 of the multi-dimensional map DRW1 is published time. That is, the dimensions DIM1 and DIM2 correspond to different feature labels relatively. It is noted that, the multi-dimensional map DRW1 comprises a plurality of dimensions corresponding to various types of feature labels. For example, the types of feature labels can comprise (1) published time, (2) publication, (3) drug name label, (4) symptom label, (5) author label, (6) drug receptor label, (7) dosage form label, (8) drug composition or active pharmaceutical ingredient, (9) side effect, (10) pharmacological mechanism. In this case, the corresponding multi-dimensional map DRW1 comprises 9 dimensions, and a single second medical document can be represent by an 8×1 matrix. And so forth, the multi-dimensional map DRW1 with 500 second medical documents can be represent by an 8×500 matrix. Since it is hard to fully illustrate the multi-dimensional feature of the multi-dimensional map DRW1 in the figure, the two dimensions DIM1 and DIM2 shown in FIG. 5 illustrate an example of the multi-dimensional map DRW1.

As shown in FIG. 4 and FIG. 5, the process unit 120 executes step S231, determining word vectors V1-V5 of each of the plurality of second medical documents D2a-D2e on the dimensions DIM1 and DIM2 of the plurality of feature labels by using the natural language model 124.

For example, the natural language model 124 determines the second medical document D2a has an author AUR1 (corresponding to the dimension DIM1) and a published time PUB1 (corresponding to the dimension DIM2), and generates the word vector V1 of the second medical document D2a accordingly.

The natural language model 124 determines the second medical document D2b also has the author AUR1 (corresponding to the dimension DIM1) and a published time PUB2 (corresponding to the dimension DIM2), and generates the word vector V2 of the second medical document D2b accordingly. At this point, the author AUR1 of the second medical document D2b is as same as the author AUR1 of the second medical document D2a, so the coordinate of the word vector V2 is as same as the coordinate of the word vector V1 in the dimension DIM1. On the contrary, the published time PUB2 of the second medical document D2b is later than the published time PUB1 of the second medical document D2a, so the coordinate of the word vector V2 is different from the coordinate of the word vector V1 in the dimension DIM2.

The natural language model 124 determines the second medical document D2c has another author AUR2 (corresponding to the dimension DIM1) and the published time PUB2 (corresponding to the dimension DIM2), and generates the word vector V3 of the second medical document D2c accordingly. At this point, the author AUR3 of the second medical document D2b is different from the author AUR1 of the second medical document D2b, so the coordinate of the word vector V2 is as same as the coordinate of the word vector V1 in the dimension DIM1. On the contrary, the published time PUB2 of the second medical document D2c is as same as the published time PUB2 of the second medical document D2b, so the coordinate of the word vector V3 is as same as the coordinate of the word vector V2 in the dimension DIM2.

And so forth, the natural language model 124 determines the word vector V4 and V5 relatively corresponding to the second medical documents D2d and D2e.

As shown in FIG. 4 and FIG. 5, the process unit 120 executes step S232, projecting the plurality of second medical documents D2a-D2e onto coordinate positions in the multi-dimensional map DRW1 based on the word vectors V1-V5 of each of the plurality of second medical documents D2a-D2e.

The distances between the coordinate positions correspond to a correlation between the plurality of second medical documents D2a-D2e. For example, the longer the time interval between the published times, the lower the correlation; and the shorter the time interval between the published times, the higher the correlation. In another example, the correlation is higher if the authors are the same, and the correlation is lower if the authors are different.

The second medical documents with shorter distances from each other in the multi-dimensional map DRW1 can form a high correlation group, e.g., the second medical documents D2d and D2e have the same author AUR2, and the corresponding published time PUB3 and PUB4 are close to each other. Thus, the second medical documents D2d and D2e can form a high correlation group GD2. In practice, numerous medical documents can form a plurality of high correlation groups in different areas of the multi-dimensional map DRW1 and do not limited by the high correlation group GD2 shown in FIG. 5.

As shown in FIG. 1 and FIG. 2, the process unit 120 executes step S240 subsequently, in response to selecting a first symptom, the process unit 120 estimates a plurality of second symptoms related to the first symptom. For example, the user can select a first symptom currently being studied via the user interface 180, e.g., “cough”. Corresponding to the first symptom “cough” currently selected, the process unit 120 can search the collected second medical documents by using the natural language model 124 to determine whether “cough” or other similar symptoms are mentioned in the second medical documents and estimate the second symptoms, such as “fever”, “headache”, and “runny nose”, related to the first symptom “cough” accordingly.

As shown in FIG. 1, FIG. 2, and FIG. 5, the process unit 120 executes step S250 subsequently, selecting a plurality of coordinates matching the first symptom “cough” and the plurality of second symptoms “fever”, “headache”, and “runny nose” from the multi-dimensional map DRW1, and selecting a plurality of third medical documents from the plurality of second medical documents in the multi-dimensional map DRW1 based on a node adjacent to the plurality of coordinates.

In an embodiment, the third medical documents selected may mention the first symptom “cough” and the second symptoms “fever”, “headache”, and “runny nose”; or the third medical documents may selected from the medical documents which corresponding to the coordinates closed to documents mentioning the first symptom and the second symptoms in the multi-dimensional map DRW1 so that determined high correlated with the above-mentioned documents (e.g., have the same author, have the same published time, mention the same pharmacological mechanism, or have similar active pharmaceutical ingredient), in the meantime, the first symptom and the second symptoms may not mentioned in the medical documents directly.

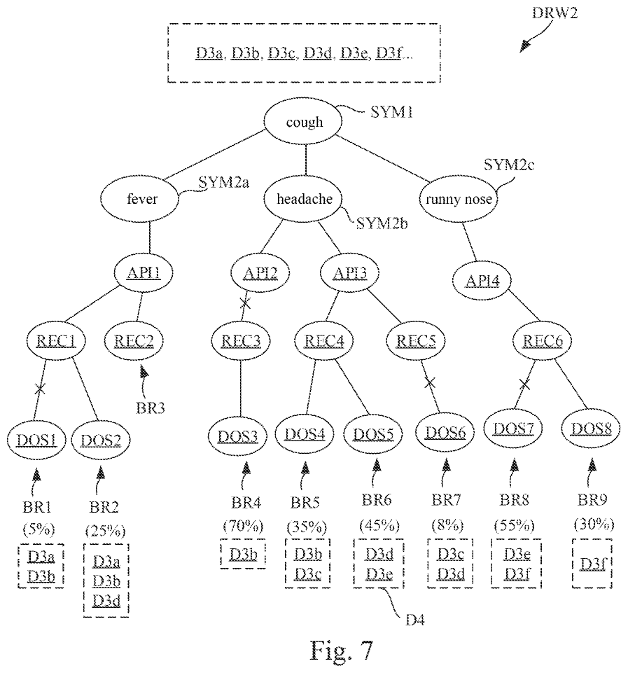

As shown in FIG. 1 and FIG. 2, the process unit 120 executes step S260 subsequently, analyzing a correlation between the plurality of third medical documents and the at least one feature label of each of the plurality of third medical documents to form a label topology map. References are also made to FIG. 6 and FIG. 7. FIG. 6 is a flow diagram illustrating detailed steps of step S260 shown in FIG. 2 according to some embodiments of the present disclosure. FIG. 7 is a schematic diagram illustrating a label topology map DRW2 according to some embodiments of the present disclosure.

In an embodiment shown in FIG. 7, the label topology map DRW2 formed based on the correlations between third medical documents D3a-D3f and the feature labels of each of the third medical documents D3a-D3f is shown. A top layer node in the label topology map DRW2 is a first symptom SYM1, and the label topology map DRW2 comprises 9 branch paths BR1-BR9, wherein each of the branch paths BR1-BR9 starts from the first symptom SYM1 and passes through second symptoms SYM2a-SYM2c, drug formulations API1-API4, receptor names REC1-REC6, and drug dosage forms DOS1-DOS8.

As shown in FIG. 6 and FIG. 7, the process unit 120 executes step S261, analyzing the selected third medical documents D3a-D3f by using the natural language model 124 to obtain the above-mentioned drug formulations, receptor names, and drug dosage forms and forming the label topology map DRW2 with the branch paths BR1-BR9 accordingly.

For example, the feature labels of each of the third medical documents D3a, D3b, and D3d are related to the first symptom SYM1 “cough” and the second symptom SYM2a “fever” and adopt the related content and experimental result of the drug formulation API1, the receptor name REC1, and the drug dosage form DOS2. Therefore, the process unit 120 can analyze the third medical documents D3a, D3b, and D3d by using the natural language model 124 to obtain the branch path BR2.

In another example, the feature labels of each of the third medical documents D3d and D3e are related to the first symptom SYM1 “cough” and the second symptom SYM2b “headache” and adopt the related content and experimental result of the drug formulation API3, the receptor name REC4, and the drug dosage form DOS5. Therefore, the process unit 120 can analyze the third medical documents D3d and D3e by using the natural language model 124 to obtain the branch path BR6.

That is, the process unit 120 executes the natural language model 124 to classify the feature labels of each of the third medical documents D3a-D3f and obtain the branch path BR1-BR9. Generally, massive time is needed if the third medical documents D3a-D3f are studied and classified by personnel. In this embodiment, the third medical documents D3a-D3f is classified into the branch path BR1-BR9 automatically by analyzing the feature labels of each of the third medical documents D3a-D3f. The different third medical documents D3a-D3f can be quickly classified into different development paths (i.e., corresponding to the branch paths BR1-BR9) without manpower involved.

The branch paths BR3 in the label topology map DRW2 shown in FIG. 7 is not connected to any drug dosage form, and it may represent that in the case where the third medical documents D3a-D3f are related to the first symptom SYM1 “cough” and the second symptom SYM2a “fever” and adopt the related content and experimental result of the drug formulation API3, the receptor name REC4, and the drug dosage form DOS5, none of the drug dosage form is mentioned in the third medical documents D3a-D3f.

In an embodiment, step S261 generates the branch path BR1-BR9 in the label topology map DRW2, and the medical document analysis system 100 and the medical document analysis method 200 can further analyze the development potential and value of each of the branch path BR1-BR9.

As shown in FIG. 6 and FIG. 7, the process unit 120 executes step S262, analyzing an experimental result mentioned in each of the third medical documents D3a-D3f selected, and the experimental result comprises a drug effect, a drug antagonism, or a drug side effect.

The process unit 120 executes step S263, selecting the third medical documents D3a-D3f with a corresponding feature label for the first symptom, the drug formulation, the receptor name to the drug dosage form of each of the branch paths BR1-BR9 in the label topology map DRW2, and performing a strategy neural network simulation or a value neural network simulation by using the neural network 126 to obtain a simulation success rate of each of the branch paths BR1-BR9. For example, the neural network 126 can adopt a Monte Carlo simulation algorithm, an Ant Colony Optimization (ACO), or a Genetic Algorithm (GA) to perform the strategy neural network simulation or the value neural network simulation.

For example, the third medical documents D3a and D3b correspond to the branch path BR1, accordingly, the process unit 120 estimates the simulation success rate of the branch path BR1 by using the neural network 126 based on an experimental result (i.e., a drug effect, a drug antagonism, or a drug side effect) mentioned in the third medical documents D3a and D3b. In this case, since the experimental result corresponding to the branch path BR1 mentioned in the third medical documents D3a and D3b is relatively negative (e.g., the side effect is relatively obvious), the simulation success rate estimated by using the neural network 126 may be 5%.

In another example, the third medical documents D3a, D3b and D3d correspond to the branch path BR2, accordingly, the process unit 120 estimates the simulation success rate of the branch path BR2 by using the neural network 126 based on an experimental result (i.e., a drug effect, a drug antagonism, or a drug side effect) mentioned in the third medical documents D3a, D3b and D3d. In this case, since the experimental result corresponding to the branch path BR2 mentioned in the third medical documents D3a, D3b and D3d is relatively neutral (e.g., the medication is moderately effective in reducing symptoms and no significant side effects are mentioned), the simulation success rate estimated by using the neural network 126 may be 25%.

In another example, the third medical documents D3d and D3e correspond to the branch path BR6, accordingly, the process unit 120 estimates the simulation success rate of the branch path BR6 by using the neural network 126 based on an experimental result (i.e., a drug effect, a drug antagonism, or a drug side effect) mentioned in the third medical documents D3d and D3e. In this case, since the experimental result corresponding to the branch path BR6 mentioned in the third medical documents D3d and D3e is relatively positive (e.g., the medication is effective in reducing symptoms), the simulation success rate estimated by using the neural network 126 may be 45%.

The process unit 120 executes step S264, determining whether one of the simulation success rates estimated of the branch paths is lower than a threshold (in this embodiment, assume that the threshold is set as 10%). In response to the simulation success rate of any branch path in the label topology map DRW2 being lower than 10%, determining the corresponding branch path to be invalid.

In the embodiment shown in FIG. 7, since the simulation success rates of the branch paths BR1 and BR7 are 5% and 8% respectively, the branch paths BR1 and BR7 are determined to be invalid in step S264.

As shown in FIG. 6 and FIG. 7, the process unit 120 executes step S265, taking the first symptom, the second symptom, the drug formulation, the receptor name, and the drug dosage form passed by each of the branch paths BR1-BR9 in the label topology map DRW2 as a patent search query, and searching in the patent database DB2 by using the crawler 122. By this, whether a valid patent corresponding to the search condition of each of the branch paths BR1-BR9 exists in the patent database DB2 is determined.

The process unit 120 executes step S266, in response to a valid patent corresponding to any branch path being searched from the patent database DB2, determining the corresponding branch path in the label topology map DRW2 to be invalid. For example, in the embodiment shown in FIG. 7, since the first symptom SYM1, the second symptom SYM2b, the drug formulation API2, the receptor name REC3, and the drug dosage form DOS3 passed by the branch path BR4 are taken as a patent search query, and a valid patent applied by a company is searched by the patent search query in the patent database DB2, the branch path BR4 is determined to be invalid in step S266. Additionally, since the first symptom SYM1, the second symptom SYM2c, the drug formulation API4, the receptor name REC6, and the drug dosage form DOS7 passed by the branch path BR8 are taken as a patent search query, and a valid patent applied by a company is also searched by the patent search query in the patent database DB2, the branch path BR8 is determined to be invalid in step S266.

Step S265 and S266 can prevent the developers from investing their budget and attention in technology protected by others' patents during development, so as to prevent the drugs developed in the future from being unable to be produced, manufactured or sell, and it can also invest development resources on technology with greater commercial potential.

In this embodiment, after the invalid branch paths BR1, BR4, BR7 and BR8 in the label topology map DRW2 shown in FIG. 7 are excluded, step S270 can be executed based on the currently valid branch paths.

As shown in FIG. 1 and FIG. 2, the process unit 120 executes step S270, selecting a target branch path from the plurality of branch paths, and displaying information related to the target branch path.

In some embodiments, during the process of selecting the target branch path, first, the process unit 120 sorts the valid branch paths BR2, BR3, BR5, BR6, and BR9 based on each of their simulation success rates, and next, select the target branch path based on the sorted branch paths. In the embodiment shown in FIG. 7, the branch path BR6 has the highest simulation success rate 45% in the valid branch paths and can be selected as the target branch path in some embodiments.

A single branch path is selected as the target branch path, but the present disclosure is not limited thereto. In other embodiments, the branch path BR6 and the branch path BR5 with the highest simulation success rate and the second highest simulation success rate can be selected as the target branch paths at the same time.

In this embodiment, the process unit 120 selects the third medical documents D3d and D3e corresponding to the target branch path BR6 as fourth medical documents D4, and displays the fourth medical documents D4 in the user interface 180. Therefore, the user can check the fourth medical documents D4 (including the third medical documents D3d and D3e) related to the target branch path (i.e., the branch path BR6) in the user interface 180.

Next, the user can read the fourth medical documents D4 on the target branch path first, and carry out research and development subsequently. Additionally, the fourth medical documents D4 can also indicate the possibility of different drug dosage forms in present drug ingredient. For example, a certain drug ingredient is marketed in a dosage form A and has patent protection, by using the medical document analysis method 200 mentioned above, a dosage form B of the drug ingredient not yet been developed may be found. In this way, the user can develop the dosage form B based on the drug ingredient, and apply for patent protection.

On the other hand, in the process of the process unit 120 selecting the target branch path, commercial value of each of the branch paths can be further considered. Namely, the process unit 120 evaluates the commercial values of each of the valid branch paths BR2, BR3, BR5, BR6, and BR9 first. For example, the commercial values of each of the branch paths BR2, BR3, BR5, BR6, and BR9 can be calculated based on sales records or patient populations of the drugs mentioned by each of the branch paths BR2, BR3, BR5, BR6, and BR9.

Next, the process unit 120 sorts the valid branch paths BR2, BR3, BR5, BR6, and BR9 based on their respective weighted simulation success rates and weighted commercial values and select the target branch path based on the sorted branch paths. In this case, since the commercial value of the branch path BR5 may be larger than the commercial value of the branch path BR6, the branch path BR5 is selected as the target branch path instead, the third medical documents D3b and D3c corresponding to the branch path BR5 are selected as the fourth medical documents D4 instead, and the fourth medical documents D4 are displayed in the user interface 180.

The medical document analysis method 200 mentioned above can be fully executed multiple times and find out the development direction with the highest commercial value, the highest potential, or relatively fast development.

For example, a certain disease may have N symptoms, each of the symptoms may have M blocking method (e.g., interference, replacement, virus and bacteria replicating prevention, or receptor site), there are P existing drugs that can achieve blockade, and the existing drugs may have Q ingestion methods (e.g., oral, injection, patch, inhalation, suppository and other drug dosage forms). Therefore, in order to treat the N symptoms of the disease, there may be M×N×P×Q branch paths, wherein some of the S branch paths may have been determined to be difficult to succeed by medical documents, and T branch paths may have been patented, and R branch paths may have obvious side effects recorded in medical documents, thus, possible development directions can be found by using the medical document analysis method 200, in theory, M×N×P×Q−S−R−T different branch paths can be found. The medical document analysis method 200 in the present disclosure can assist in finding the various branch paths mentioned above, and give a better development direction quickly, that is, find out the target branch path with greater commercial potential or higher success rate.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A medical document analysis method, comprising:
performing a recursive search based on keywords of a plurality of first medical documents to obtain a plurality of second medical documents;
analyzing contents of the plurality of second medical documents, and marking each of the plurality of second medical documents with a plurality of feature labels;
projecting the plurality of second medical documents onto different coordinate positions in a multi-dimensional map respectively based on at least one of the plurality of feature labels of each of the plurality of second medical documents, wherein dimensions of the multi-dimensional map correspond to the plurality of feature labels, and distances between the coordinate positions correspond to a correlation between the plurality of second medical documents
in response to selecting a first symptom, estimating a plurality of second symptoms related to the first symptom;
selecting a plurality of coordinates matching the first symptom and the plurality of second symptoms from the multi-dimensional map, and selecting a plurality of third medical documents from the plurality of second medical documents based on a node adjacent to the plurality of coordinates;
analyzing a correlation between the plurality of third medical documents and the at least one of the plurality of feature labels of each of the plurality of third medical documents in the multi-dimensional map to form a label topology map, wherein a top layer node in the label topology map is the first symptom, the label topology map comprises a plurality of branch paths, and each of the plurality of branch paths starts from the first symptom and passes through a drug formulation, a receptor name and a drug dosage form; and
selecting a target branch path from the plurality of branch paths, and displaying information related to the target branch path.

2. The medical document analysis method of claim 1, wherein the step of performing the recursive search based on the keywords of the plurality of first medical documents comprises:
(a1) selecting a plurality of keywords from the plurality of first medical documents by using a natural language model;
(a2) searching for a plurality of related medical documents based on the plurality of keywords;
(a3) determining whether the plurality of related medical documents meet a convergence condition;
(a4) in response to the convergence condition not being met, updating the plurality of keywords by extracting the plurality of related medical documents by using the natural language model, and back to the step (a3); and
(a5) in response to the convergence condition being met, taking the plurality of related medical documents as the plurality of second medical documents.

3. The medical document analysis method of claim 2, wherein after the step (a1), further comprises:
adding a new keyword to the plurality of keywords, or removing a excluded keyword from the plurality of keywords; and
assigning a plurality of weights to each of the plurality of keywords;
wherein the step (a2) further comprises:
searching for the plurality of related medical documents based on the plurality of weighted keywords.

4. The medical document analysis method of claim 1, wherein the plurality of feature labels in the step of marking each of the plurality of second medical documents with the plurality of feature labels comprise a published time, a publication, a drug name label, a symptom label, an author label, a drug receptor label, and a dosage form label.

5. The medical document analysis method of claim 1, wherein the step of projecting the plurality of second medical documents onto the different coordinate positions in the multi-dimensional map respectively comprises:
(b1) determining a word vector of each of the plurality of second medical documents on dimensions of the plurality of feature labels by using a natural language model; and
(b2) projecting the plurality of second medical documents onto the multi-dimensional map based on the word vector of each of the plurality of second medical documents.

6. The medical document analysis method of claim 1, wherein the step of forming the label topology map based on the correlation between the plurality of third medical documents and the at least one of the plurality of feature labels of each of the plurality of third medical documents comprises:
(c1) analyzing an experimental result mentioned in each of the plurality of third medical documents selected, and the experimental result comprises a drug effect, a drug antagonism, or a drug side effect;
(c2) selecting the plurality of third medical documents with a corresponding feature label for the first symptom, the drug formulation, the receptor name to the drug dosage form of each of the plurality of branch paths in the label topology map, and performing a strategy neural network simulation or a value neural network simulation to obtain a simulation success rate of each of the plurality of branch paths; and
(c3) in response to the simulation success rate of one of the plurality of branch paths in the label topology map being lower than a threshold, determining the one of the plurality of branch paths to be invalid.

7. The medical document analysis method of claim 6, wherein the step of forming the label topology map based on the correlation between the plurality of third medical documents and the at least one of the plurality of feature labels of each of the plurality of third medical documents comprises:
(c4) searching in at least one patent database for the first symptom, the drug formulation, the receptor name, and the drug dosage form passed by each of the plurality of branch paths in the label topology map; and
(c5) in response to a valid patent corresponding to the one of the plurality of branch paths being searched from the at least one patent database, determining the one of the plurality of branch paths in the label topology map to be invalid.

8. The medical document analysis method of claim 7, wherein the step of selecting the target branch path from the plurality of branch paths comprises:
sorting the plurality of valid branch paths based on the simulation success rate of each of the plurality of valid branch paths; and
selecting the target branch path based on the plurality of sorted branch paths.

9. The medical document analysis method of claim 7, wherein the step of selecting the target branch path from the plurality of branch paths comprises:
estimating a commercial value of each of the plurality of valid branch paths;
sorting the plurality of valid branch paths based on the simulation success rate and the commercial value of each of the plurality of valid branch paths; and
selecting the target branch path based on the plurality of sorted branch paths.

10. The medical document analysis method of claim 1, wherein the step of displaying the information related to the target branch path further comprises:
selecting at least one fourth medical documents related to the target branch path from the plurality of third medical documents; and
displaying the at least one fourth medical documents.

11. A medical document analysis system, comprising:
a communication transceiver unit, configured to communicatively connected to an academic database;
a user interface; and
a process unit, coupled to the communication transceiver unit and the user interface, and the process unit is configured to:
performing a recursive search based on keywords of a plurality of first medical documents to obtain a plurality of second medical documents;
analyzing contents of the plurality of second medical documents, and marking each of the plurality of second medical documents with a plurality of feature labels;
projecting the plurality of second medical documents onto different coordinate positions in a multi-dimensional map respectively based on at least one of the plurality of feature labels of each of the plurality of second medical documents, wherein dimensions of the multi-dimensional map correspond to the plurality of feature labels, and distances between the coordinate positions correspond to a correlation between the plurality of second medical documents;
in response to selecting a first symptom, estimating a plurality of second symptoms related to the first symptom;
selecting a plurality of coordinates matching the first symptom and the plurality of second symptoms from the multi-dimensional map, and selecting a plurality of third medical documents from the plurality of second medical documents based on a node adjacent to the plurality of coordinates;
analyzing a correlation between the plurality of third medical documents and the at least one of the plurality of feature labels of each of the plurality of third medical documents in the multi-dimensional map to form a label topology map, wherein a top layer node in the label topology map is the first symptom, the label topology map comprises a plurality of branch paths, and each of the plurality of branch paths starts from the first symptom and passes through a drug formulation, a receptor name, and a drug dosage form; and
selecting a target branch path from the plurality of branch paths, and displaying information related to the target branch path.

12. The medical document analysis system of claim 11, wherein the operation of performing the recursive search based on the keywords of the plurality of first medical documents comprises:
(a1) selecting a plurality of keywords from the plurality of first medical documents by using a natural language model;
(a2) searching for a plurality of related medical documents based on the plurality of keywords;
(a3) determining whether the plurality of related medical documents meet a convergence condition;
(a4) in response to the convergence condition not being met, updating the plurality of keywords by extracting the plurality of related medical documents by using the natural language model, and back to the operation (a3); and
(a5) in response to the convergence condition being met, taking the plurality of related medical documents as the plurality of second medical documents.

13. The medical document analysis system of claim 12, wherein after the operation (a1), further comprises:
adding a new keyword to the plurality of keywords, or removing a excluded keyword from the plurality of keywords; and
assigning a plurality of weights to each of the plurality of keywords;
wherein the operation (a2) further comprises:
searching for the plurality of related medical documents based on the plurality of weighted keywords.

14. The medical document analysis system of claim 11, wherein the plurality of feature labels in the operation of marking each of the plurality of second medical documents with the plurality of feature labels comprise a published time, a publication, a drug name label, a symptom label, an author label, a drug receptor label, and a dosage form label.

15. The medical document analysis system of claim 11, wherein the operation of projecting the plurality of second medical documents onto the different coordinate positions in the multi-dimensional map respectively comprises:
(b1) determining a word vector of each of the plurality of second medical documents on dimensions of the plurality of feature labels by using a natural language model; and
(b2) projecting the plurality of second medical documents onto the multi-dimensional map based on the word vector of each of the plurality of second medical documents.

16. The medical document analysis system of claim 11, wherein the operation of forming the label topology map based on the correlation between the plurality of third medical documents and the at least one of the plurality of feature labels of each of the plurality of third medical documents comprises:
(c1) analyzing an experimental result mentioned in each of the plurality of third medical documents selected, and the experimental result comprises a drug effect, a drug antagonism, or a drug side effect;
(c2) selecting the plurality of third medical documents with a corresponding feature label for the first symptom, the drug formulation, the receptor name to the drug dosage form of each of the plurality of branch paths in the label topology map, and performing a strategy neural network simulation or a value neural network simulation to obtain a simulation success rate of each of the plurality of branch paths; and
(c3) in response to the simulation success rate of one of the plurality of branch paths in the label topology map being lower than a threshold, determining the one of the plurality of branch paths to be invalid.

17. The medical document analysis system of claim 16, wherein the operation of forming the label topology map based on the correlation between the plurality of third medical documents and the at least one of the plurality of feature labels of each of the plurality of third medical documents comprises:
(c4) searching in at least one patent database for the first symptom, the drug formulation, the receptor name, and the drug dosage form passed by each of the plurality of branch paths in the label topology map; and
(c5) in response to a valid patent corresponding to the one of the plurality of branch paths being searched from the at least one patent database, determining the one of the plurality of branch paths in the label topology map to be invalid.

18. The medical document analysis system of claim 17, wherein the operation of selecting the target branch path from the plurality of branch paths comprises:
sorting the plurality of valid branch paths based on the simulation success rate of each of the plurality of valid branch paths; and
selecting the target branch path based on the plurality of sorted branch paths.

19. The medical document analysis system of claim 17, wherein the operation of selecting the target branch path from the plurality of branch paths comprises:
estimating a commercial value of each of the plurality of valid branch paths;
sorting the plurality of valid branch paths based on the simulation success rate and the commercial value of each of the plurality of valid branch paths; and
selecting the target branch path based on the plurality of sorted branch paths.

20. The medical document analysis system of claim 11, wherein the operation of displaying the information related to the target branch path further comprises:
selecting at least one fourth medical documents related to the target branch path from the plurality of third medical documents; and
displaying the at least one fourth medical documents.
